(19) United States
(12) Patent Application Publication
(10) Pub. No.: US 2009/0175917 A1
(43) Pub. Date: Jul. 9, 2009

(54) Tooth Bleaching Agent

(75) Inventors: Jüergen Engelbrecht, Hamburg (DE); K.J. Gorlich, Elmshorn (DE)

Correspondence Address: EDWARDS ANGELL PALMER & DODGE LLP, P.O. BOX 55874, BOSTON, MA, 02205, US

(73) Assignee: S&C Polymer, Silicon- und Composite Spezialitaten GmbH, Elmshorn (DE)

(21) Appl. No.: 12/226,251

(22) PCT Filed: Apr. 13, 2007
(86) PCT No.: PCT/EP2007/003301

§ 371 Date: Oct. 10, 2008

(30) Foreign Application Priority Data
Apr. 13, 2006 (DE) 10 2006 017 814.9

(51) Int. Cl. A61K 8/21; A61Q 11/00; A61K 8/46; A61K 8/02; A61K 8/22

(52) U.S. Cl. 424/401; 424/52; 424/57; 424/53

(57) ABSTRACT

The invention relates to “tooth-bleaching agent” compositions and to their applications in whitening teeth. The dental bleaching agent of the present invention comprises apatite, especially preferably in particle sizes in the nano range, likewise especially preferably in the form of fluorapatite. The tooth-bleaching agent can have a desensitising action and a positive action in respect of the remineralisation of the tooth surface.

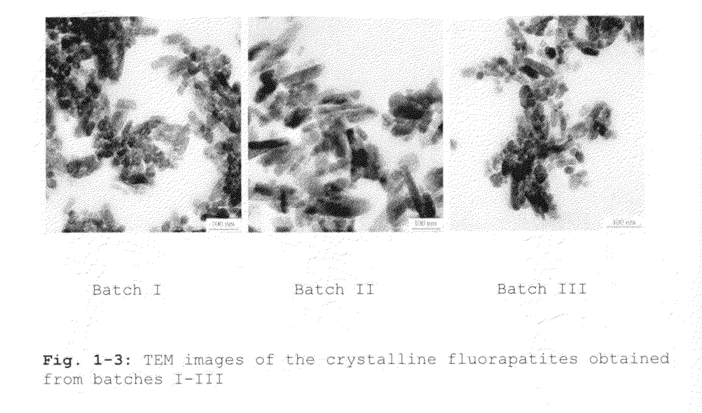

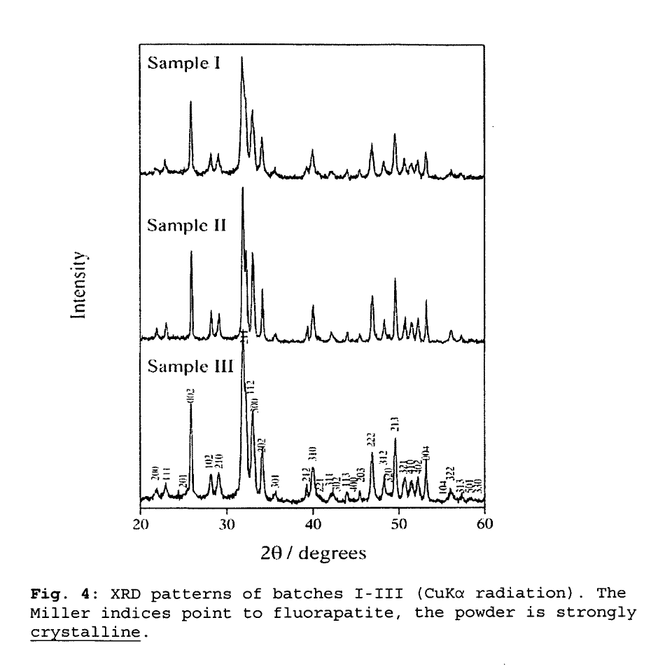

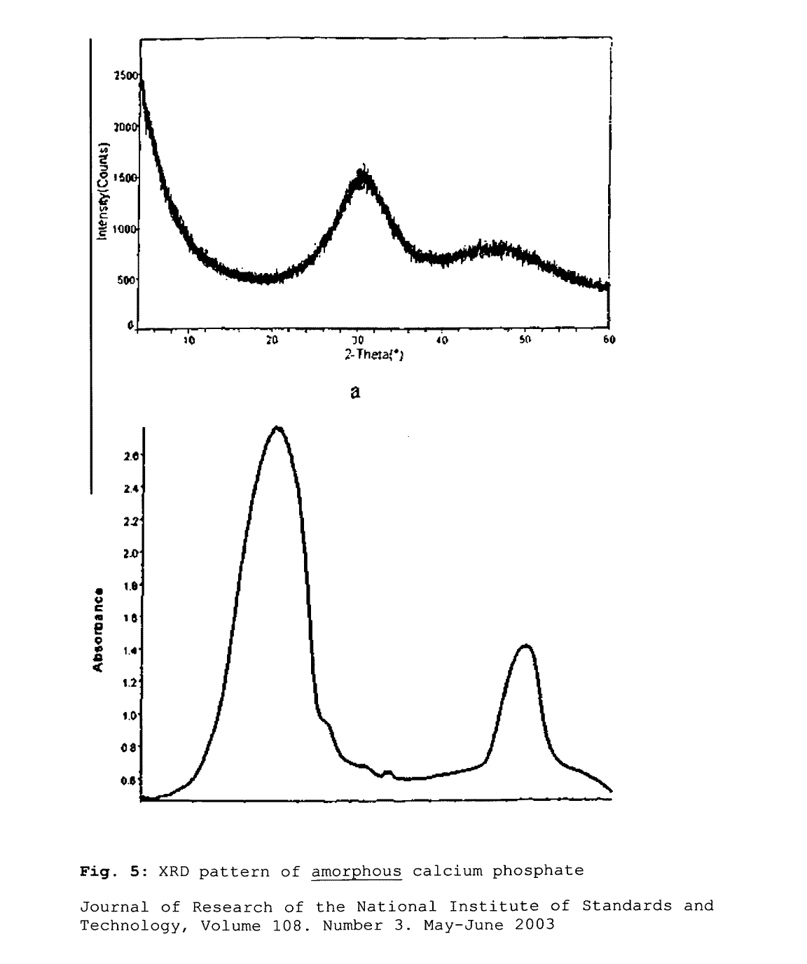

The invention relates to “tooth-bleaching agent” compositions and to their applications in whitening teeth. The dental bleaching agent of the present invention comprises apatite, especially preferably in particle sizes in the nano range, likewise especially preferably in the form of fluorapatite. The tooth-bleaching agent can have a desensitising action and a positive action in respect of the remineralisation of the tooth surface.

### PRIOR ART

The discoloration of teeth may be caused by the natural ageing process, by the consumption of certain foodstuffs and tobacco, by diseases, by injuries, by medicaments and by inherited and environmental conditions. Since white or light-coloured teeth are generally perceived as being more aesthetically pleasing than dark or discoloured teeth, there has always been great interest in the development of materials and methods for whitening teeth.

Some tooth-cleaning agents, such as toothpastes, dental gels and tooth powders, contain bleaching materials that release active oxygen or hydrogen peroxide. Such bleaching agents comprise peroxides, percarbonates and perborates of alkali metals and alkaline earth metals or complex compounds containing hydrogen peroxide.

One of the bleaching materials most often used in dentistry is percarbamide, also known as urea peroxohydrate or urea hydrogen peroxide. Percarbamide has been used as an oral antiseptic in dentistry for decades. Urea itself is described in the literature as a keratinisation agent for the gums. Tooth-bleaching was observed to be a side-effect when contact times were prolonged. Other bleaching agents, such as, for example, peroxyacetic acid and sodium perborate, are likewise well known in the fields of medicine, dentistry and cosmetics.

The bleaching gels available on the market, which are also referred to worldwide by the English term “bleaching gels”, are divided into three categories (Reality Report Vol. 14/2000), namely “Power Bleaching”, “Assisted Bleaching” and “Home Bleaching”. In order to save time and expense, in dental practice “Power Bleaching” is the preferred method of bleaching discoloured teeth.

U.S. Pat. No. 5,098,303 (Fischer, 1992), U.S. Pat. No. 5,234,342 (Fischer, 1993), U.S. Pat. No. 5,376,006 (Fischer, 1994), U.S. Pat. No. 5,725,843 (Fischer, 1998), U.S. Pat. No. 5,746,598 (Fischer, 1998), U.S. Pat. No. 5,759,038 (Fischer, 1998), U.S. Pat. No. 5,770,105 (Fischer, 1998), U.S. Pat. No. 5,785,527 (Fischer et al., 1998), U.S. Pat. No. 5,858,332 (Fischer et al., 1999), WO Pat. No. 9,937,236 (Fischer et al., 1999), U.S. Pat. No. 5,985,249 (Fischer, 1999), U.S. Pat. No. 6,036,943 (Fischer, 2000), WO Pat. No. 0,028,953 (Fischer et al., 2000), U.S. Pat. No. 6,086,855 (Fischer, 2000) and U.S. Pat. No. 6,183,251 (Fischer, 2001) describe tooth-bleaching methods and tooth-bleaching or fluoride gels which comprise as active agent hydrogen peroxide, percarbamide, sodium perborate, benzoyl peroxide, glycerol peroxide, and as additives water, glycerol, propylene glycol, polyethylene glycol, erythritol, sorbitol, mannitol, carboxypolymethylenes, thickeners, such as xanthan, talha, tragacanth, locust bean flour, guar, ghatti, furcellaran, carrageenan, alginic acids, agar, alginates, proteins, desensitising substances, fluorides such as sodium monofluorophosphate, sodium fluoride and zinc fluoride, anti-microbial substances, such as chlorohexidine, tetracycline, cetyl pyridinium chloride, benzalkonium chloride, cetyl pyridinium bromide, methyl benzoate and propyl benzoate.

The company Discus Dental (USA) sells bleaching agents having additives of amorphous calcium triphosphate. Their advantage lies in the presence of the additive, which is suitable principally for remineralisation, during the bleaching process. However, in slightly acidic conditions, in which the above-mentioned bleaching agents are generally relatively stable, they are not stable. They also do not contain any fluoride.

PCT patent WO 02/49578 A1 describes a remineralising dental adhesive film consisting of a support material, which adheres to the tooth, and active ingredients embedded therein. The active ingredients consist of hydroxyapatite, fluorapatite, calcium fluoride and dicalcium, tricalcium or tetracalcium phosphate.

An important objective of the present invention was the provision of a new and improved single-component or multi-component bleaching agent for teeth that has desensitising, fluoride-releasing and remineralising properties.

### DESCRIPTION OF THE INVENTION

According to the invention there is provided a tooth-bleaching agent which comprises an apatite of the general composition

Ca₁₀₋ₓMₓ(PO₄)_{6-y}B_{y}A_{z}(OH)_{2-z}

wherein M is a cation other than Ca²⁺, B is an anion other than PO₄³⁻,

A is selected from the group consisting of O²⁻, CO₃²⁻, F⁻ and Cl⁻ and the following relationships apply to the parameters x, y, z: 0≦x≦9, 0≦y≦5 and 0≦z≦2, preferably with the proviso that the sum of the charges of the cations corresponds to the sum of the charges of the anions. The mentioned numbers can also take the form of fractions. In preferred embodiments, the apatite is characterised in that more than 50% by weight of the apatite particles have a particle size in the range of <500 nm, especially preferably in the range of <200 nm and more especially preferably in the range of <100 nm. According to the invention, such apatites are also referred to as nano-apatites.

In addition to the apatite, the tooth-bleaching agent of this invention comprises at least one orally compatible bleaching agent. Various bleaching agents and/or bleaching agent mixtures (also referred to as peroxides and peroxide mixtures, respectively) can be used for the preparation of the tooth-bleaching agent, such as, for example, hydrogen peroxide, percarbamide, sodium perborate, potassium peroxymonosulfate, potassium chlorate, potassium percarbonate, sodium percarbonate, calcium peroxide, magnesium peroxide, perphosphates, persilicates, benzoyl peroxide, glycerol peroxide, calcium hydrogen carbonate peroxide and sodium hydrogen carbonate peroxide, with preference being given to hydrogen peroxide, percarbamide, sodium perborate and potassium peroxymonosulfate. Hydrogen peroxide, percarbamide, sodium perborate and/or potassium peroxymonosulfate and/or mixtures thereof are present in the total tooth-bleaching agent preferably in an amount of from 5 to 70% by weight, especially in an amount of from 5 to 55% by weight.

The content of the bleaching agent in the total tooth-bleaching agent can be from 5 to 75% by weight, preferably from 5 to 60% by weight, especially from 10 to 30% by weight, more especially from 15 to 25% by weight.

The tooth-bleaching agent of this invention can also comprise one or more activator components. The activator component may be a gel, for example an alkaline gel. It preferably contains one or more alkali metal and/or alkaline earth metal salts. As activators or decomposition catalysts it is possible to select salts or complexes especially from the group copper, manganese and/or iron, more especially organometal complexes or salts such as, for example, acetylacetonates, gluconates, lactates, fumarates, naphthenic acid salts, metallocenes, oxalates, citrates, sulfates, oxides, acetates and/or mixtures thereof. In accordance with their chemical character they exhibit violent to mild reactions during the decomposition of the peroxides. In the case of alkaline gels, preference is given to pH values of from 8 to 12; in the case of metal complexes and salts it is preferable to use amounts of from 0.01 to 10% by weight, especially preferably amounts of from 0.05 to 5% by weight, based on the total tooth-bleaching agent. The activator component can additionally also comprise a different peroxide.

The content of activator component in the total tooth-bleaching agent can be from 0.1 to 30% by weight, preferably from 0.2 to 20% by weight, especially from 0.5 to 10% by weight.

Optionally, the bleaching agent and/or the optional activator component can contain gel-formers or thickeners. Examples are cellulose polymers, polycarboxylic acids, pyrogenic silicon dioxide, poly(meth)acrylic acids, polysaccharides, polyvinyl butyrals, alginates, cumarone resins, shellac, xanthan, tragacanth, guar, carrageenan, alginic acids etc. and/or mixtures thereof. They can be present together in an amount of from 0.01 to 20% by weight, preferably in an amount of from 0.05 to 15% by weight.

As base material for the preparation of stable tooth-bleaching agents there is often used water or water in combination with other base materials. Such base materials comprise or consist of polyols such as polyethylene glycol, sorbitol, polypropylene glycol, propylene glycol, glycerol, ethanol, acetone, ether, acetates, xylitol and others and/or mixtures of those mentioned. Polyols such as glycerol and/or propylene glycol and/or demineralised water are preferred in this invention. They are present either on their own or as mixtures and are present in an amount of from 0.1 to 98% by weight, and preferably in an amount of from 0.5 to 95% by weight, based on the total tooth-bleaching agent.

Further constituents may be present, such as, for example, stabilisers such as alkali metal polyphosphates, alkali metal pyrophosphates, ethylene diamine tetraacetic acid and salts thereof, tartaric acid and salts thereof, citric acid and salts thereof, gluconic acid and salts thereof, triethanolamine, tin nitrate, adipic acid, tin phosphate, succinic acid etc., such as, for example, constituents that alter the pH value such as alkali metal and alkaline earth metal salts, such as, for example, vitamins as anti-inflammatories, as well as flavourings such as, for example, peppermint, vanilla etc., colorants for colouring purposes and as indicators, preservatives, fluoride derivatives, wetting agents etc. They can be present in the bleaching agents of this invention both on their own and in mixtures.

The bleaching agents, in addition to or instead of one or more activators, may also be activated by the action of heat (mouth temperature, hot light, lasers or optionally other sources), optionally supported by additives of energy-absorbing substances such as, for example, carotenoids, coronene, bixin, perylene, flavins, etc.

It is known that apatites are an important base material for the embedding of calcium in the hard substances of the tooth (for example enamel, dentine, bone) and that, combined with other phosphate-containing and non-phosphate-containing minerals, they play an important role in respect of healthy teeth. The best known representative of that class of substance is hydroxyapatite having the stoichiometric formula Ca₁₀(PO₄)₆(OH)₂ or Ca₅(PO₄)₃OH. In its synthetic and biocompatible form it is used for a large number of applications in dentistry, orthopaedics and oral surgery, but it never occurs in its pure form in biological tissue. That is a result of the possible isomorphic exchange of the Ca²⁺, PO₄³⁻ and OH⁻ ions. The Ca²⁺ ion can be replaced by a number of (mostly divalent) cations. The phosphate anion, on the one hand, can be replaced by carbonate, hydrogen phosphate, pyrophosphate, sulfate, aluminate and silicate ions, while, on the other hand, the hydroxide ion can be replaced by halide, carbonate and oxide ions.

Of that large number of possible naturally occurring or synthesised apatites, hydroxyapatite is the one most often used for the preparation of materials for orthodontics or for biometric applications.

Although the problem has already been discussed in the literature, little is known about the exchange of cations in apatite. It is known that the degree of order in the crystallising solid increases as the size of the exchange cation increases. In theory it is not possible to predict the possible extent of the replacement by chemically or crystallographically similar cations, but it has been found that the method of synthesis of the hydroxyapatite has a crucial effect on future exchange potential. The following, in particular, come into consideration as possible exchange cations:

Na⁺, K⁺, Mg²⁺, Ca²⁺, Sr²⁺, Ba²⁺, Y²⁺, Ti²⁺, Zr²⁺, Mn²⁺, Fe²⁺, Pd²⁺, Cu²⁺, Ag⁺, Zn²⁺, Sn²⁺, Re²⁺, Re³⁺, Al³⁺, In³⁺ and/or Y³⁺.

For example, about 30% of the calcium content can be replaced by strontium without the crystalline structure's being altered. The presence of that element in apatites used in the field of dentistry is significant against the background of a possible caries-inhibiting effect and reduced dentine sensitivity. Moreover, solubility is reduced.

Furthermore, it has been found in the context of the invention that an anti-microbial action is achievable by incorporation of certain cations into apatites. Particularly advantageous in this connection is the incorporation of Cu²⁺, Ag²⁺, Zn²⁺ and/or Sn²⁺.

It is also possible for anions to act as exchange ions. As B anion, special mention should be made of CO₃²⁻, HPO₄²⁻, HCO₃⁻ and P₂O₇⁴⁻, the y value usually being 0-2. The hydroxyapatite is characterised by the absence of A or z=0, while fluorapatite is obtained when A=F and z=2.

In fluorapatite and in chlorapatite the hydroxide ions have been replaced by fluoride and chloride ions, respectively. Fluorapatite is characterised by an increase in crystal dimensions and a reduction in the parameters of the elementary cell. Furthermore, its solubility is lower and its thermal stability is greater, for which reason it is used in the treatment of bone diseases or dental caries. In comparison with fluorapatite, in chlorapatite the cell parameter a of the elementary cell is increased and the cell parameter c is reduced. The different crystal lattice is a result of the different ion radii of the fluoride and chloride.

Fluorapatite is of particular interest. By virtue of its relatively low solubility in the weakly acidic range, fluoridation of the tooth surfaces, that is to say conversion of hydroxyapatite into fluorapatite at the surface of the tooth, can result in teeth that are less open to attack by acid and are therefore more resistant to caries.

The presence of fluorapatite on the surface of the tooth gives rise to the possibility of ion exchange between the hydroxyapatite of the tooth surface and the fluorapatite during the bleaching time. The surface of the tooth is accordingly cleaned by oxidation and at the same time rendered more resistant to acid. In particular, because the bleaching agent for use in a tooth-bleaching agent generally has a slightly acidic pH in order to ensure the stability of the bleaching agent, such as, for example, a peroxide, the tooth enamel undergoes slight etching during the bleaching. The simultaneous presence of the apatite results, surprisingly, in immediate, that is to say in situ, repair of the attacked tooth enamel during the bleaching. Because the specific surface area of nanoparticles is especially large, the remineralising action of the nano-apatites can also be orders of magnitude greater.

The nano-apatites, which preferably consist of or comprise nanoparticles, can be produced by the methods usually employed for the production of nanocrystalline materials, for example by (i) atom-based methods (chemical or physical vapour deposition, condensation in the gaseous phase, reactions from aerosols) or (ii) by conventional methods (mechanical abrasion, crystallisation from the amorphous phase, phase separation).

There are also a large number of possible methods of producing nanocrystalline materials from clusters which are prepared by chemical or physical methods. For example, the precipitation method is a conventional method of producing very fine-grained powders or colloidal suspensions which are successfully used for the synthesis of clusters in the nano range, for example in sol-gel technology. Furthermore, it is also possible to produce clusters in nano dimensions or nano-structured powders having relatively large particle dimensions by means of gas reactions in the high-temperature range (R. W. Siegel, 1991, loc. cit.).

Nanocrystalline materials are generally synthetically produced materials which are characterised by continuous phases or by granular structures and a length of usually less than 200 nm. In dependence upon the number of dimensions in which those materials have a nanostructure, a distinction is made between (i) zero-dimensional materials (atom clusters, for example dispersed in a matrix of non-nanocrystalline material; threads, tubuli), (ii) one-dimensional materials (mono-molecular layers that are nanodimensioned only in respect of the layer thickness), (iii) two-dimensional materials (granular superposed layers, “granular superpositions”, ultra-fine layers) and (iv) three-dimensional materials (structures that are nanoscale in all three dimensions) (R. W. Siegel, in Materials Science and Technology, Vol. 15: Processing of Metals and Alloys, R. W. Chan, 583 (1991)).

The specific properties of the nanocrystalline materials result from three basic features, namely (i) the atomic size range of ≦200 nm, (ii) the high proportion of atoms participating in the interfaces and (iii) the interactions between the individual sub-regions.

In the case of particle sizes in the nano range there is a high proportion of surface molecules in relation to the total number of molecules of a particle. In a material having an average particle size of 10-15 nm, from 15 to 50% of the atoms participate in the “particle interfaces”.

Because the number of interfaces in nanocrystalline materials is very much higher than in conventional materials, by suitable control in the course of the synthesis of the materials the nature of the interactions between the interfaces of all phases involved can be affected by the nature of the interfaces.

The unusual nature of the surface interactions combined with low solubility make nano-apatites appear especially suitable as an additive according to the invention in bleaching agents: the interactions of crystalline nano-apatite in a bleaching formulation with its biological environment can be far more intensive than in the case of customary apatite. Nano-apatite crystals remaining in attacked (bleached) interstitial spaces, for example between tooth enamel prisms or in dentine tubulis, can have a desensitising and remineralising action over a prolonged period.

The particle size of the apatites to be used according to the invention in the dental material is not critical over the ranges indicated. In preferred embodiments, they are characterised in that more than 50% by weight, optionally more than 60% by weight or even more than 70% by weight of the apatite particles have a particle size in the range of <500 nm, especially preferably more than 50% by weight, optionally more than 60% by weight or even more than 70% by weight of the apatite particles have a particle size in the range of <200 nm, and very especially preferably more than 50% by weight, optionally more than 60% by weight or even more than 70% by weight of the apatite particles have a particle size in the range of <100 nm.

In a preferred embodiment of the invention, the apatite particles have been surface-treated in order to achieve better dispersivity.

For example, the apatite fillers can have been surface-treated with esters of phosphoric, phosphonic or carboxylic acids. Special preference is given to the esters of mono-, di- and tri-phosphonic acids, such as, for example, tris(phosphono-methyl)amine, azacycloheptan-2,2-diphosphonic acid, hydroxy-ethane-1,1-diphosphonic acid. Furthermore, treatments with phosphate salts may be advantageous. Also suitable as surface-treatment agents are water-soluble anionic, cationic or amphoteric surfactants, polymeric protective colloids, such as, for example, polyvinyl alcohol, polyacrylic acids, polyvinylpyrrolidone, etc., as well as polyethylene glycol acids.

A further method is the application of a SiO₂ or ZrO₂ layer on a nanometre scale and subsequent treatment with a functional silane such as, for example, hydroxy, amino or alkyl organo-silanes.

According to the invention, the apatites are contained in the bleaching material in an amount sufficient to allow exchange of ions with the biological environment. Preference is given to amounts by weight of from 1 to 20% by weight, especially from 2 to 10%, based on the total weight of the bleaching material.

In addition to the essential and characteristic component nano-apatite, the bleaching material can have additions of optional, but nevertheless very advantageous and no less preferred desensitising agents which are likewise able to support the desensitising and remineralising action in the bleaching agent, such as, for example, fluorides (sodium monofluorophosphate, sodium fluoride, calcium fluoride etc.), nitrates (sodium nitrate, potassium nitrate etc.), strontium compounds (e.g. strontium chloride etc.).

As further additives it is also possible for other bioactive or antibiotic substances, without any kind of limitation, to be added, such as, for example, transforming growth factor-beta, cell-attachment factors, endothelial growth factors, bone morphogenetic proteins, penicillin, chlortetracycline hydrochloride, chloramphenicol, oxytetracycline etc.

The tooth-bleaching agent according to the invention may also comprise wetting agents as further additives, preference being given to the use of sodium lauryl sulfate.

By virtue of their excellent remineralising and desensitising properties, the bleaching agents described according to the invention can be used very satisfactorily in the whitening of teeth.

The apatite content of the tooth-bleaching formulation can release ions (inter alia fluoride, phosphate, calcium) onto the tooth being bleached.

On the other hand, in the case of nanocrystallinity and in view of the low solubility of the apatite, especially fluorapatite, when nanocrystals remain in the interstitial gaps in the enamel or dentine a remineralising action going beyond the bleaching process can be provided.

The effect sought according to the invention, namely whitening of the teeth combined with simultaneous repair of very small lesions as a result of the exchange of ions with the tooth substance and, especially when nano-fluorapatite is used, the “hardening” of tooth enamel as a result of fluoride exchange, is achieved by the tooth-bleaching formulations according to the invention.

For example, the apatite additive according to the invention can be used in typical dental bleaching formulations, such as glycerol as base material, pyrogenic silicon dioxide as thickener, percarbamide as bleaching agent.

The invention will be described in greater detail below with reference to exemplary embodiments and comparison examples.

### Example 1

### Preparation of Nanocrystalline Calcium Fluorapatite

Nanocrystalline fluorapatite was crystallised from a ternary microemulsion. For that purpose, an aqueous phase comprising CaCl₂ (Merck, Darmstadt, Germany) was emulsified into a mixture of Empilan KB6ZA (ethoxylated lauryl alcohol, Albright & Wilson, Meuse, France) and octane (Sigma-Aldrich, Schnelldorf, Germany) in a fixed ratio of 3:7. The microemulsion was stirred vigorously at 30° C. with 30% by weight (I), 36.36% by weight (II) and 50% by weight (III) 1.0M CaCl₂ in order to obtain a microemulsion. Again with vigorous stirring, a stoichiometric amount of an aqueous solution containing 0.6 mol of Na₂HPO₄ and 0.2 mol of KF (Merck) was added and the mixture was left to stand for 24 hours at 30° C. The powder was isolated by centrifugation and washed twice with alcohol and once with water, each time with isolation by centrifugation. Freeze-drying was then carried out for 48 hours.

The ultra-fine powders were examined in respect of crystallinity, morphology and particle size. High-resolution TEM images showed defined crystallites, mostly rod-shaped. The particle sizes were 20-130 nm. X-ray diffractometer patterns show a high degree of crystallinity.

TABLE 1
Diameter and length of the apatite crystallite
particles (Batch I-III) from the TEM images.

| Batch | Diameter [nm] | Length [nm] |
|---|---|---|
| I | 28 | 84 |
| II | 29 | 127 |
| III | 23 | 52 |

The SEM-EDX data (energy dispersive X-ray spectrometry) of the powder show sufficiently good agreement with calcium fluorapatite, see also Figures.

| | | | | | |
|---|---|---|---|---|---|
| | | | | | Ca/P |
| Batch | Mol % O | Mol % F | Mol % P | Mol % Ca | molar ratio |

Ca—F apatite (calc.)

| | | | | | |
|---|---|---|---|---|---|
| | 57.1 | 4.8 | 14.3 | 23.8 | 1.66 |
| I | 60.64 | 5.41 | 13.67 | 20.28 | 1.48 |
| II | 59.79 | 5.67 | 14.26 | 20.28 | 1.42 |
| III | 56.87 | 4.96 | 15.40 | 22.77 | 1.48 |

All three samples show the characteristic bands of calcium fluorapatite in the IR spectrum.

IR wave number [cm⁻¹]/allocation

3426/OH stretching vibration, 1638 w/H₂O water of crystallisation, 1099 vs/u₃ PO₄ antisym.,

1038 vs/u₃ PO₄ antisym., 965 w/u₁ PO₄ sym., 868 w/CO₃ stretching vibration

606 S/u₄ PO₄, 567 S/u₄ PO₄, 474 w/u₂ PO₄, 326 S/u₃ Ca₃—F “sublattice mode”,

273/u₃ Ca—PO₄ “lattice mode”, 229/u₃ Ca—PO₄ “lattice mode”

FT-IR data of Ca₁₀(PO₄)₆F₂.

Relative intensities: vs=very strong, s=strong, m=medium, w=weak

### Example 2

### Surface Modification of the Nano-Apatite

100 g of nano-apatite powder from Example 1 (I) is made into a slurry in acetone and, with constant stirring, 6 g of hydroxyethyl phosphoric acid ester are added thereto. After 2 hours' stirring, centrifugation and washing three times with acetone, drying was carried out.

### Examples 3

### Single-Component Gels

Glycerol was used as base material and pyrogenic silicon dioxide as thickener.

### Comparison Example

| | | |
|---|---|---|
| a) | glycerol | 73.00% w/w |
| | thickener | 5.00% w/w |
| | percarbamide | 22.00% w/w |

Example according to the invention:

| | | |
|---|---|---|
| b) | glycerol | 64.00% w/w |
| | thickener | 4.00% w/w |
| | percarbamide | 22.00% w/w |
| | nano-fluorapatite | 10.00% w/w |
| | (according to Example 1) | |

The prepared bleaching formulations were used for tooth-whitening on enamel and dentine.

Whereas in the case of Example a) sensitisation occasionally occurred, no oversensitisation whatsoever was observed even on repeated application of the tooth-bleaching agent according to the invention to sensitive areas, such as dentinal areas.

### Examples 4

### Two-Component Gels

Glycerol was used as base material and pyrogenic silicon dioxide as thickener.

### Comparison Example

| | | |
|---|---|---|
| Component I: | glycerol | 83.00% w/w |
| | thickener | 6.00% w/w |
| | percarbamide | 11.00% w/w |

| | | |
|---|---|---|
| Component II: | glycerol | 93.00% w/w |
| | thickener | 6.40% w/w |
| | Fe(II) sulfate | 0.60% w/w |

Example according to the invention:

| | | |
|---|---|---|
| Component I: | glycerol | 74.00% w/w |
| | thickener | 5.00% w/w |
| | percarbamide | 11.00% w/w |
| | nano-fluorapatite | 10.00% w/w |
| | (according to Example 1) | |

| | | |
|---|---|---|
| Component II: | glycerol | 93.00% w/w |
| | thickener | 6.40% w/w |
| | Fe(II) sulfate | 0.60% w/w |

Here too, the result was analogous to the previous result. The formulation according to the invention was as good as nonsensitising.

1-26. (canceled)

27. A tooth-bleaching agent comprising an apatite of the formula:
Ca₁₀₋ₓMₓ(PO₄)_{6-y}B_{y}A_{z}(OH)_{2-z}
wherein M is a cation other than Ca²⁺, B is an anion other than PO₄³⁻, A is selected from the group consisting of O²⁻, CO₃²⁻, F⁻ and Cl⁻ and the following relationships apply to the parameters x, y and z: 0≦x≦9, 0≦y≦5 and 0≦z≦2, and a bleaching agent.

28. A tooth-bleaching agent of claim 27 wherein x and y are 0, the anion A is F⁻, and z=2 (fluorapatite).

29. A tooth-bleaching agent of claim 27 wherein x, y and z are 0 (hydroxyapatite).

30. A tooth-bleaching agent of claim 27 wherein each M, independently of any other(s), is Mg²⁺, Sr²⁺, Ba²⁺, Y²⁺, Ti²⁺, Zr²⁺, Mn²⁺, Fe²⁺, Pd²⁺, Cu²⁺, Ag⁺, Zn²⁺, Sn²⁺, Re³⁺, Re²⁺, Al³⁺, In³⁺, Y³⁺, Na⁺ and/or K⁺.

31. A tooth-bleaching agent of claim 27 wherein more than 50% by weight of the apatite is present in a particle size of <500 nm.

32. A tooth-bleaching agent of claim 27 wherein more than 50% by weight of the apatite is present in a particle size of <200 nm.

33. A tooth-bleaching agent according to claim 27 wherein more than 50% by weight of the apatite is present in a particle size of <100 nm.

34. A tooth-bleaching agent according to claim 27 wherein the apatite has been surface-treated.

35. A tooth-bleaching agent according to claim 27 wherein the amount by weight of apatite is from 1 to 20% by weight, based on the total tooth-bleaching agent.

36. A tooth-bleaching agent according to claim 27 wherein the amount by weight of apatite is from 2 to 10% by weight, based on the total tooth-bleaching agent.

37. A tooth-bleaching agent according to claim 27 wherein the agent comprises at least one further desensitising agent.

38. A tooth-bleaching agent according to claim 27 wherein the bleaching agent has a pH value of from 2 to 7.

39. A tooth-bleaching agent according to claim 27 wherein the bleaching agent comprises hydrogen peroxide and/or percarbamide and/or sodium perborate and/or potassium peroxymonosulfate and/or mixtures thereof.

40. A tooth-bleaching agent according to claim 27 wherein the bleaching agent(s) is(are) present in an amount of from 5 to 70% by weight based on the total bleaching agent.

41. A tooth-bleaching agent according to claim 27 wherein the agent further comprises an activator component.

42. A tooth-bleaching agent according to claim 41 wherein the activator component comprises at least one alkaline additive.

43. A tooth-bleaching agent according to claim 41 wherein the alkaline additive is one or more alkali and/or alkaline earth metal salts.

44. A tooth-bleaching agent according to claim 41 wherein the activator component comprises at least one salt or complex from the group copper, manganese and iron.

45. A tooth-bleaching agent according to claim 44 wherein the salt or the complex is an acetylacetonate, gluconate, lactate, fumarate, naphthenic acid salt, metallocene, oxalate, citrate, sulfate, oxide, acetate and/or mixtures thereof.

46. A tooth-bleaching agent according to claim 44 wherein the salt or the complex is present in an amount of from 0.01 to 10% by weight, based on the total tooth-bleaching agent.

47. A tooth-bleaching agent according to claim 44 wherein the salt or the complex is present in an amount of from 0.05 to 5% by weight, based on the total tooth-bleaching agent.

48. A tooth-bleaching agent according to claim 27 wherein, in addition to comprising at least one apatite, it also comprises at least one other bioactive substance.

49. A tooth-bleaching agent according to claim 27 wherein the agent further comprises at least one antibiotic substance.

50. A tooth-bleaching agent according to claim 27 wherein the agent further comprises at least one energy-absorbing substance.

51. A tooth-bleaching agent according to claim 27 wherein the agent further comprises at least one wetting agent.

52. A tooth-bleaching agent according to claim 27 wherein the agent further comprises at least one stabiliser and/or at least one preservative and/or at least one colorant and/or at least one flavouring.
